SEQUENCE LISTING (14 sequences)

SEQ ID NO: 1 - protein, 295 aa, Artificial Sequence
  Synthetic
TFGSGEADCGLRPLFEKKSLEDKTERALLESYIAGRIVAGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 2 - protein, 295 aa, Artificial Sequence
  Synthetic
TFGSGEADCGLRPLFEKKSLEDKTERALLESYIAGRIVAGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSAGAGCDRDGKYGFYTHVFRLKKWIQKVIDQFGE

SEQ ID NO: 3 - protein, 295 aa, Artificial Sequence
  Synthetic
TFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 4 - protein, 308 aa, Artificial Sequence
  Synthetic
TATSEYQTFFNPRTFGSGEADCGLRPLFEKKSLEDKTERELLESYIDGRIVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 5 - protein, 308 aa, Artificial Sequence
  Synthetic
TATSEYQTFFNPRTFGSGEADCGLRPLFEKKSLEDKTERALLESYIAGRIVAGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 6 - protein, 461 aa, Artificial Sequence
  Synthetic
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 7 - protein, 419 aa, Artificial Sequence
  Synthetic
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 8 - protein, 419 aa, Artificial Sequence
  Synthetic
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTADQEDQVAPRLIAGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 9 - protein, 6 aa, Artificial Sequence
  Synthetic
IDGRIV

SEQ ID NO: 10 - protein, 6 aa, Artificial Sequence
  Synthetic
IDPRIV

SEQ ID NO: 11 - DNA, 46 bp, Artificial Sequence
  Synthetic
cacagcagaccaagaagaccaagtagctccgcggctcattgctggg

SEQ ID NO: 12 - DNA, 46 bp, Artificial Sequence
  Synthetic
cccagcaatgagccgcggagctacttggtcttcttggtctgctgtg

SEQ ID NO: 13 - DNA, 34 bp, Artificial Sequence
  Synthetic
tgcctgcgagggcgacgctggggggcccatggtc

SEQ ID NO: 14 - DNA, 34 bp, Artificial Sequence
  Synthetic
gaccatgggccccccagcgtcgccctcgcaggca

(19) United States
(12) Patent Application Publication
(10) Pub. No.: US 2016/0152964 A1
(43) Pub. Date: Jun. 2, 2016

(54) RECOMBINANT AUTO-ACTIVATING PROTEASE PRECURSORS

(71) Applicant: Saint Louis University, St. Louis, MO (US)

(72) Inventors: Nicola Pozzi, St. Louis, MO (US); Enrico Di Cera, Ladue, MO (US); Sergio Barranco-Medina, St. Louis, MO (US)

(21) Appl. No.: 15/004,280

(22) Filed: Jan. 22, 2016

Related U.S. Application Data
(63) Continuation of application No. 13/473,566, filed on May 16, 2012, now abandoned.

(51) Int. Cl. C12N 9/74

(52) U.S. Cl. CPC C12N 9/6429; C12Y 304/21005

(57) ABSTRACT

A recombinant serine protease precursor that auto-activates in an aqueous buffer to form a mature active enzyme is disclosed. A contemplated precursor contains 1 to about 10 heterologous amino acid residues that function to enhance by at least ten-fold the room temperature rate of auto-lytic bond cleavage to form the active enzyme relative to the auto-lytic cleavage rate of the native enzyme precursor when each precursor is dispersed in an aqueous buffer at an optimal pH value for the proteolytic activity of the protease. Illustrative active enzymes include serine proteases such as thrombin and protein C. A method of preparing and using an enzyme precursor is also disclosed.

### CROSS REFERENCE TO RELATED APPLICATIONS

This application is a continuation application of U.S. patent application Ser. No. 13/473,566, filed on May 16, 2012, which is hereby incorporated by reference in its entirety.

### GOVERNMENTAL SUPPORT

The present invention was made with governmental support under grants HL049413, HL058141, HL073813 and HL095315 awarded by the National Institutes of Health. The government has certain rights in the invention.

### INCORPORATION OF SEQUENCE LISTING

A paper copy of the Sequence Listing and a computer readable form of the Sequence Listing containing the file named “SLU 11-031(3003528-0109)_ST25.txt”, which is 26,046 bytes in size (as measured in MICROSOFT WINDOWS® EXPLORER), are provided herein and are herein incorporated by reference. This Sequence Listing consists of SEQ ID NOs:1-14.

### TECHNICAL FIELD

The invention relates to the field of recombinant proteins, and particularly to auto-activating recombinant proteases. The invention provides recombinant protease precursors that auto-activate, thereby providing the active form of the protease.

### BACKGROUND OF THE INVENTION

Proteases are naturally occurring enzymes that are crucial for the regulation of many aspects of physiology and pathology, including blood coagulation, wound-healing, immune responses, reproduction, and digestion. Too much or too little activity of a particular protease is a hallmark of many diseases. Compounds that inhibit the catalytic function of overabundant proteases are suitable drug candidates for treatment. When genetic or pathological disorders result in inadequate amounts of particular proteases in the body due to decreased production, activity or accelerated breakdown, periodic administration of the active protease is essential for life. In each scenario, significant quantities of the protease are required for effective treatment of protease-related disorders.

Nearly all proteases are synthesized as zymogens, that are inactive precursors of the active enzyme. The conversion from zymogen to active enzyme is usually a multi-step process resulting in the removal/cleavage of part of the zymogen form, which then exposes or activates the mature enzyme's proteolytic site.

Usually, zymogens are in a latent state and cannot initiate this multi-step conversion process without participation by other enzymes. However, after the conversion process has been initiated by other enzymes, some intermediate precursor forms can participate in subsequent steps of the conversion process.

Four protease families by themselves account for over 40% of all proteolytic enzymes in humans. These are the ubiquitin-specific proteases responsible for regulated intracellular protein turnover, the adamalysins that control growth factors and integrin function and include the metalloproteases, prolyl oligopeptidases, and the trypsin-like serine proteases, which are also the largest group of homologous proteases in the human genome [DiCera, (2009) IUBMB Life. 61(5):510-515].

Serine proteases represent the most abundant family of proteolytic enzymes and are crucial for many aspects of physiology and pathology. The family members are expressed as inactive zymogens that are irreversibly converted to mature, active proteases by a series of proteolytic cleavages. Serine proteases play a central role in digestion, blood coagulation, fibrinolysis, development, fertilization, apoptosis and immunity.

The serine protease thrombin is a physiological component of blood coagulation and wound healing. Thrombin is synthesized in the liver as the inactive precursor prothrombin. The zymogen circulates in the blood at a concentration of 0.1-0.2 mg/ml. In nature, the conversion of prothrombin into thrombin is regulated by a multicomponent system, known as prothrombinase complex, that is formed by Factor Xa, Factor Va, phospholipid, and calcium ions. Alternatively, prothrombin is converted to active thrombin by treatment with ecarin, a metalloprotease present in snake venom of Echis carinatus. Either way, an exogenous protease (such as Factor Xa or ecarin) is absolutely required to catalyze this reaction [Morita et al., (1980) Meth. Enz. 80:303-311; Speijer et al., (1986) J. Biol. Chem. 261:13258-13267; and Yonemura et al., (2004) J. Biochem. 135:577-582].

The serine protease-activated protein C (aPC) is another physiological component of blood coagulation and wound healing. aPC is synthesized as an inactive precursor, called protein C, and is converted into activated protein C by a multistep process requiring other enzymes. [U.S. Pat. No. 5,831,025; U.S. Pat. No. 5,330,907; U.S. Pat. No. 4,908,314]. Mature, active thrombin is one of the enzymes involved in the activation of protein C. [U.S. Pat. No. 5,831,025].

Similarly, the serine protease chymotrypsin, an enzyme involved in digestion, is initially synthesized as the inactive zymogen chymotrypsinogen. After initial cleavage by trypsin, the resulting intermediate form completes the conversion process by removing additional portions of itself.

All of these proteases are initially synthesized as inactive forms that lack the ability to cleave other proteins or themselves. Thereby, complete activation occurs after a complex, usually multistep, process that is initiated by another enzyme. After the conversion process is initiated, some intermediate protease forms can participate in later steps of the process.

Once active, many serine proteases are used in clinical applications. Thrombin variants exhibit anticoagulant and antithrombotic activity both in vitro and in vivo [Arosio et al., (2000) Biochemistry 39:8095-8101; Cantwell et al., (2000) J. Biol. Chem. 275:39827-39830; Berny et al., (2008) Arterioscler, Thromb. Vasc. Biol. 18:329-334; Feistritzer, (2006) J. Biol. Chem. 281:20077-20084; Gruber et al., (2002) J. Biol. Chem. 277:27581-27584; Gruber et al., (2006) J. Thromb. Haemost. 4:392-397; Gruber et al., (2007) Blood 109:3733-3740].

Thrombin variants provide a potent and safe antithrombotic effect by blocking the interaction of von Willebrand Factor with the platelet receptor GpIb [Berny et al., (2008) Arterioscler, Thromb. Vasc. Biol. 18:329-334; Gruber, (2007) Blood 109:3733-3740]. Activated protein C variants offer cytoprotective advantages [Feistritzer et al., (2006) J. Biol. Chem. 281:20077-20084]. Chymotrypsin has been used clinically as an anti-inflammatory agent and for debridement of necrotic tissue from ulcers, burns, and wounds. [Prueter et al., (1957) Can. Med. Assoc. 76:1040-1043].

Because of their participation in numerous physiological, biological, and chemical processes, considerable effort has been devoted to the production and isolation of active proteases. Thrombin was initially obtained as prothrombin isolated from human or animal plasma and then activated by the prothrombinase complex or snake venom proteases.

Because plasma-derived products carry an inherent risk of disease transmission, recombinant human thrombin has been pursued as an alternative that would reduce that risk of disease transmission. U.S. Pat. No. 6,413,737 B1 described new forms of recombinant ecarin, a prothrombin-specific protease isolated from snake venom and methods of producing active thrombin by exposing prothrombin to recombinant ecarin. U.S. Pat. No. 8,062,876 B2 described a method of activating thrombin by passing an aqueous solution of prethrombin-1 over oscutarin-C, another prothrombin protease isolated from snake venom, immobilized on a solid support.

Efforts to produce protease zymogens as recombinant proteins have enjoyed some success. [U.S. Pat. No. 6,420,157; U.S. Pat. No. 5,858,758]. Recombinant human proteases have been expressed and produced in animal cell cultures; for example, recombinant thrombin has been produced in Chinese hamster ovary cells. [U.S. Pat. No. 8,062,876 B2]. However, maintenance and propagation of animal cell lines is complicated and expensive.

There remains a need in the art for methods of efficient and inexpensive delivery of thrombin, activated protein C, and other active proteases, free from traditional biological and chemical contaminants.

### BRIEF SUMMARY OF THE INVENTION

The present invention, contemplates an auto-activating recombinant protease precursor (zymogen) molecule that is comprised of at least two sequence portions. A first sequence portion is an amino acid residue sequence of an active protease, including the active site, in which the sequence is at least 95 percent identical to that of a wild type or native protease. The second sequence portion contains 2 to about 200 residues and comprises a so-called activation peptide.

The two sequence portions are joined by peptide bonds to at least one linking target amino acid residue sequence up to eight residues in length that has the amino acid residue sequence and scissile bond of a cleavage site (the target sequence) split by the native protease. When dispersed in an aqueous buffer and maintained at room temperature and at an optimal pH value for the protease, the first polypeptide sequence portion cleaves the scissile bond of the target amino acid residue sequence in the absence of other enzymes.

The protease precursor includes at least one heterologous residue that functions to enhance the room temperature rate of auto-lytic scissile bond cleavage by at least ten-fold relative to the auto-lytic cleavage rate of the native enzyme precursor when each precursor is dispersed in a room temperature aqueous buffer at an optimal pH value for the protease.

The number of heterologous residues that function to enhance the rate of autolysis can be up to about 10 residues, but is more preferably about 1 to about 6 residues. A heterologous residue can be present in the target sequence, in another region or regions of the protein, or in both.

The recombinant protease precursor thus reacts with itself to form an active protease. Thus, a contemplated recombinant protease precursor or zymogen can be said to “auto-activate” to form an active protease, or to be auto-lytic.

A preferred protease is a serine protease. A more preferred serine protease is a trypsin-like serine protease that cleaves the polypeptide chain on the carboxyl side (following) of a positively charged amino acid residue such as lysine or arginine.

One preferred aspect contemplates a recombinant protease precursor, such as a thrombin EDE precursor of SEQ ID NO:1 or a thrombin EDEWE precursor of SEQ ID NO:2. A contemplated precursor molecule can be expressed in bacterial cells that do not glycosylate the expressed protein, or mammalian cells that do glycosylate the protein. Bacterially-expressed, glycosylation-free, e.g., Escherichia coli culture-derived or -expressed, as well as mammalian cell-expressed or -derived thrombin EDE and thrombin EDEWE precursors are also contemplated that contain the SEQ ID NO:1 or SEQ ID NO:2 amino acid sequences, respectively.

Another preferred aspect contemplates a recombinant protease precursor, such as a thrombin EDE precursor of SEQ ID NO:1 or a thrombin EDEWE precursor of SEQ ID NO:2. A contemplated precursor molecule is preferentially expressed in baby hamster kidney (BHK) cells. A mammalian-expressed thrombin EDE and thrombin EDEWE precursors are also contemplated that contain the SEQ ID NO:1 or SEQ ID NO:2 amino acid sequences, respectively.

In another aspect, the invention contemplates an activated protein C precursor that contains the SEQ ID NO:8 amino acid residue sequence and is preferentially expressed in BHK cells. A mammalian-expressed activated protein C precursor is also contemplated that contains the SEQ ID NO:8 amino acid sequence.

Another aspect of the invention contemplates a pharmaceutical composition that contains an effective amount of bacteria-expressed or mammalian-expressed recombinant protease precursor dissolved or dispersed in a pharmaceutically acceptable carrier. In one embodiment, a contemplated composition is adapted to be administered parenterally. One such contemplated carrier is an isotonic aqueous buffer.

A contemplated composition is intended for therapeutic use for enhancing hemostasis or treating and preventing thrombosis. An illustrative treatment comprises administering an above composition of the above-described recombinant thrombin EDE precursor to a mammal in need of treatment for thrombosis. Another illustrative treatment comprises administering the above composition of an above-described recombinant activated protein C precursor to a mammal in need in need of treatment for thrombosis. It is contemplated that such administration is repeated a plurality of times.

A method of preparing a serine protease as described above and elsewhere herein is also contemplated. In accord with that method, a recombinant serine protease precursor as discussed above and elsewhere is dissolved or dispersed in an aqueous buffer to form a composition, with the aqueous buffer being at an optimal pH value for the protease. The composition is maintained for a time sufficient for the recombinant serine protease precursor to cleave itself and form the recombinant serine protease. The protease so prepared can be used without further isolation and purification, or can be recovered and purified to a desired extent.

### DEFINITIONS

A classification system for all known proteolytic enzymes has been developed, classifying these enzymes by similarities in their sequences and structures. This classification system is catalogued as the MEROPS database. [See Rawlings et al. (2010) Nucl. Acids Res. 38:D227-D233; See also merops.sanger.ac.uk/cgi-bin/family index?type=P]. The serine proteases are listed in families S1-S75.

As used herein, “zymogen” means an enzymatically inactive precursor of an active protease. Portions of the zymogen must be enzymatically cleaved/removed by a different enzyme to generate the mature, active form of the protease.

“Precursor” means any intermediate form a protease enzyme can adopt after the initial enzymatic processing of the zymogen, but before it achieves its final, mature, enzymatically active state. Most precursors possess some enzymatic activity and can cleave a usual target amino acid residue sequence for that protease.

The term “active enzyme” is used herein to name the protein formed when a target sequence of a precursor is cleaved. For example, thrombin is the active enzyme formed when the zymogen or precursor prothrombin-2 is cleaved. Similarly, trypsin and chymotrypsin are the active enzymes formed when their zymogens trypsinogen and chymotrypsinogen, respectively, are cleaved.

“Protease” means a mature, enzymatically active molecule that cleaves a particular peptide bond in an amino acid residue sequence at a greater rate than the hydrolytic rate in a buffer at a given pH value, and preferably cleaves with high specificity.

“Serine proteases” are a set of homologous enzymes that cleave peptide bonds and contain a nucleophilic serine amino acid at their active sites. In the MEROPS database, the families 51 through S75 contain known serine proteases.

The serine protease family of enzymes can itself be subdivided by the type of residue located at the carboxyl side of the amide bond (the P₁ position) that is cleaved by the enzyme. The hydrophobicity and shape complementarity between the peptide substrate P₁ side-chain and the enzyme S₁ binding cavity accounts for the substrate specificity of this enzyme. The serine proteases are typically categorized into three families: the chymotrypsin-like, the trypsin-like and the elastase-like enzymes.

“Chymotrypsin” (EC 3.3.21.2) is a digestive enzyme that can perform proteolysis. Chymotrypsin preferentially cleaves peptide amide bonds where the carboxyl side of the amide bond (the P₁ position) is a tyrosine, tryptophan, or phenylalanine. These amino acids contain an aromatic ring in their side-chain that fits into a ‘hydrophobic pocket’ (the S₁ position) of the enzyme.

Chymotrypsin also hydrolyzes other amide bonds in peptides at slower rates, particularly those containing leucine, tyrosine, phenylalanine, methionine, tryptophan, glycine, and asparagine amino acids at the P₁ position within a polypeptide.

Chymotrypsin is synthesized in the pancreas as a zymogen called chymotrypsinogen that is enzymatically inactive. The human zymogen is referred to as chymotrypsinogen B (EC 3.3.21.1), and contains 263 amino acid residues, including an 18-residue signal peptide. Trypsin cleaves the remaining 245-residues into three chains referred to as the chymotrypsin B chain A (residues 19-31), chain B (residues 34-164) and chain C (residues 167-263). The resulting molecule contains five disulfide bonds, with bonds Cys¹⁹Cys¹⁴⁰ and cys¹⁵⁴Cys²¹⁹ linking the three chains. Cleaved chymotrypsinogen molecules can activate each other by removing two small peptides in a trans-proteolysis. The resulting molecule is active chymotrypsin, a three-polypeptide molecule interconnected via disulfide bonds.

The term “chymotrypsin” is used herein regardless of its origin; that is, both human and non-human chymotrypsins can be used within the present invention.

Trypsin-like serine proteases cleave peptide chains whose P₁ position residues are arginine or lysine. Exemplary trypsin-like serine proteases include trypsin, thrombin, activated protein c, coagulation factor VIIa, coagulation factor IXa, coagulation factor Xa, coagulation factor XIa, coagulation factor XIIa, plasmin, acrosin, kallikrein, tissue kallikrein, complement factor D, venobin A, venobin A B, tryptase, scutelarin, kexin, u-plasminogen activator. These enzymes are part of the family of serine proteases that were also known by their EC family numbers EC 3.4.21.-.

Elastase-type serine proteases cleave peptides whose P₁ position residues are smaller and uncharged. Illustrative elastase-type serine proteases include pancreatic elastase, leukocyte elastase, pancreatic elastase 11, and pancreatic endopeptidase E. The elastase-type serine proteases also have EC family numbers EC 3.4.21.-.

See, Stryer, Biochemistry, 2^{nd} ed., W. H. Freeman & Co., San Francisco, (1981) pages 166-167. See, also, Enzyme Nomenclature 1992, Academic Press, New York, 1992. Thrombin is a trypsin-like serine endopeptidase (EC 3.4.21.5) that cleaves the Arg-Gly bond in fibrinogen to form fibrin. Human thrombin is naturally made in the body from a precursor polypeptide referred to herein as preprothrombin that contains a single strand of 622 amino acid residues. Cleavage of that preprothrombin provides prothrombin, that contains a sequence of C-terminal 579 amino acid residues (subject to potential allelic variation or N-terminal microheterogeneity), plus the previous N-terminal pre-sequence of 43 residues that includes a signal peptide of 24 residues at its N-terminus, and a propeptide of 19 residues bonded to the C-terminus of the signal peptide [Degen, et al. (1993) Biochemistry 22:2087-2097].

The term “thrombin” as used herein refers to a multifunctional enzyme that contains up to about 300 residues in two polypeptide chains connected by a disulfide bond that cleaves at least two of the following proteins: protein C, fibrinogen, or protease-activated receptor 1. Thrombin can act as a procoagulant by the proteolytic cleavage of fibrinogen to fibrin. Thrombin can also activate the clotting Factors V (FV), VIII (FVIII), XI (FXI) and XIII (FXIII) leading to perpetuation of clotting, and can cleave the platelet thrombin receptor, PAR-1, leading to platelet activation. Thrombin can also activate protein C.

“Activated protein C” refers to a vitamin K-dependent glycoprotein protease (EC 3.4.21.69). Protein C synthesis occurs in the liver and begins with expression of a single-chain molecule containing 461 amino acid residues that include a 32 amino acid N-terminus signal peptide preceding a propeptide.

Protein C is formed when a dipeptide of Lys¹⁹⁸ and Arg¹⁹⁹ is removed; this causes the transformation into a heterodimer that can contain N-linked carbohydrates on each chain when expressed from mammalian cells. The protein has one light chain (21 kDa) and one heavy chain (41 kDa) connected by a cystine disulfide bond between Cys¹⁸³ and Cys³¹⁹ Inactive protein C comprises 419 amino acids in multiple domains one Gla domain (residues 43-88); a helical aromatic segment (89-96); two epidermal growth factor (EGF)-like domains (97-132 and 136-176); an activation peptide (200-211); and a trypsin-like serine protease domain (212-450). The light chain contains the Gla- and EGF-like domains and the aromatic segment. The heavy chain contains the protease domain and the activation peptide. It is in this form that 85-90% of protein C circulates in the plasma as a zymogen, waiting to be activated.

The remaining protein C zymogen comprises slightly modified forms of the protein. Activation of the enzyme occurs when a thrombin molecule cleaves away the activation peptide from the N-terminus of the heavy chain. The active site contains a catalytic triad typical of serine proteases (His²⁵³ Asp²⁹⁹ and Ser⁴⁰²)

Activated protein C cleaves blood coagulation Factor Va and Factor VIIIa. The term “activated protein C” is used regardless of its origin; that is, both human and non-human activated protein C molecules can be used within the present invention.

A “native” (wild type; wt) sequence is that of the enzyme or precursor or target that is reported for the molecule in question as occurring in nature. Typically, a native sequence is that reported in the literature, and preferably that reported in the Universal Protein Resource data base, and particularly the UniProtKB/Swiss-Prot data base.

All amino acid residues identified herein are in the natural L-configuration. In keeping with standard polypeptide nomenclature, IUPAC-IUB Commission on Biochemical Nomenclature (1969) J. Biol. Chem. 243:3557-3559, abbreviations for amino acid residues are as shown in the following Table of Correspondence:

TABLE OF CORRESPONDENCE SYMBOL

| 1-Letter | 3-Letter | AMINO ACID |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| W | Trp | L-tryptophan |
| R | Arg | L-arginine |
| D | Asp | L-aspartic acid |
| N | Asn | L-asparagine |
| C | Cys | L-cysteine |

The present invention has several benefits and advantages.

One benefit is that the costly and time-consuming zymogen activation process requiring the use of activating enzymes or other external activators is not required to convert the contemplated protease precursors into active proteases.

A related advantage is that the absence of those externally provided activating moieties removes the requirement for their subsequent removal from the active proteases.

Another advantage of the present invention is that preparation of protease precursors in bacterial culture, whenever possible, lowers the possible risks of contamination with a mammalian pathogen or allergen.

A yet further benefit of the invention is that the production of protease precursors is less costly using a contemplated protease precursor as a reactant and expression from bacteria instead of using mammalian cells.

A still further advantage of the invention is that a protease precursor can be prepared in multiple cell culture systems, providing greater flexibility in adapting the contemplated invention to the numerous cell culture systems in use.

Still further benefits and advantages will be apparent to a worker of ordinary skill from the detailed description that follows.

### BRIEF DESCRIPTION OF DRAWINGS

In the drawings that form a portion of this disclosure,

FIG. 1, in three parts (FIG. 1A-FIG. 1C) illustrates the ability of thrombin mutants EDE and EDEWE to auto-activate themselves from thrombin zymogen prethrombin-2 precursors to mature, enzymatically active forms of thrombin. FIG. 1A contains a series of SDS-PAGE studies that show that prethrombin-2 mutant E14eA/D141A/E18A (EDE) exhibits evidence of auto-activation, which is not seen in the wild-type (WT) and is selectively abrogated by the additional mutation S195A (EDES). After heparin-SEPHAROSE® purification, the concentration of each protein was adjusted to 0.27 mg/ml and auto-activation was followed at room temperature for zero (lanes 1, 2), 4 (lanes 3, 4) and 90 (lanes 5, 6) hours. FIG. 1B shows the results of another SDS-PAGE study in which auto-activation is also observed when the E14eA/D141A/E18A mutation is introduced in the prethrombin-2 mutant W215A/E217A (WE) to yield the construct E14eA/D141A/E18A/W215A/E217A (EDEWE). In this case, the concentration was adjusted to 3 mg/ml and the reaction was followed at room temperature for zero (lanes 1, 2), 3 (lanes 3, 4) and 7 (lanes 5, 6) days. No evidence of auto-activation is detected for WE over the same time scale. Samples were analyzed under non-reducing (lanes 1, 3, 5) and reducing (lanes 2, 4, 6) conditions. In the case of EDE and EDEWE, the two bands pertaining to the A and B chains of the mature enzyme are easily detected under reducing conditions and conversion to thrombin is complete after 90 hours or 7 days, respectively. The chemical identities of the A and B chains were confirmed by N-terminal sequencing. Bands in the gel are labeled as follows: A and E mapped to N-terminal sequence GRGSE and refer to prethrombin-2 constructs with the T7tag from the expression vector partially cleaved and then processed during E. coli expression as reported (58, 59); B and F mapped to N-terminal sequence TFGSG and refer to prethrombin-2 with a single N-terminus starting at Tlh; C and G mapped to N-terminal sequence IVAGS and refer to the B chain of thrombin with the N-terminus 116 and the mutation E18A introduced in the EDE and EDEWE constructs; D and H mapped to N-terminal sequence TFGSG and refer to the A chain of thrombin with the N-terminus Tlh. FIG. 1C illustrates the kinetics of auto-activation of prethrombin-2 EDE monitored as percent of thrombin produced. The shape of the auto-activation curve is consistent with an autocatalytic process initiated by prethrombin-2 EDE itself and leading to complete conversion to thrombin.

FIG. 2 illustrates the kinetics of auto-activation of protein C wild type (wt) and mutated at positions in which the glutamic acid residue at position 160 and the aspartic acid residues at each of positions 167 and 172 were substituted with alanine residues (EDD; E160A/D167A/D172A) at zero time and 150 hours for the wild type and at zero, 24, 48, 72 and 150 hours for the EDD variant. This study was carried out as discussed in FIG. 1.

### DETAILED DESCRIPTION OF THE INVENTION

The present invention broadly contemplates an auto-activating (auto-lytic) protease precursor and an active enzyme prepared therefrom. One aspect of the present invention contemplates a recombinant protease precursor that contains at least 95 percent of the amino acid residue sequence of a hydrolytically active protease, including the active site and whose optimal pH value of proteolytic activity is known. The protease is preferably a serine protease, and more preferably a trypsin-like serine protease. The protease precursor also contains a second polypeptide portion that contains 2 to about 200 amino acid residues, and preferably about 10 to about 100 residues. In the serine proteases, this second polypeptide tends to be about 2 to about 50 residues long.

The two sequence portions are joined by at least one linking target amino acid residue sequence of up to eight residues having the amino acid residue sequence and scissile bond of a cleavage site (the target sequence) that is split by the native (wild type) protease such that the recombinant protease precursor reacts with itself to form an active protease.

The enzyme precursor contains at least one amino acid residue, up to about ten such residues, that is (are) heterologous to the native precursor molecule, and function to enhance the rate at which the scissile bond of the target sequence is cleaved by the native protease as is discussed hereinafter. The presence of one to about six heterologous residues is preferred.

The enhancement of rate of auto-lytic cleavage of the scissile bond of the target sequence in the precursor is at least ten-fold, and is more usually more than one hundred-fold, when measured at room temperature in an aqueous buffer at the optimal pH value for the protease. That is, a precursor that normally is unreactive over a matter of days auto-activates under those conditions to form the enzyme itself at a measurable reaction rate.

That at least one heterologous residue can be present in the target sequence, elsewhere in the precursor or at both locations. Once the target sequence is cleaved, the heterologous residue typically becomes part of the residuum of the target sequence. A contemplated recombinant protease precursor or zymogen can be said to “auto-activate” to form an active protease, or to be auto-lytic.

It is to be understood that “reacts with itself” is not meant to imply that a single molecule folds upon itself to cleave the target sequence. Although that may happen in some instances, the phrase “reacts with itself” is meant to indicate that a first molecule containing the enzyme active site and target sequence reacts with a second molecule containing the enzyme active site and target sequence, rather than a third, different molecule with a different active site and or different target sequence reacting with the target sequence portion.

Following usual cleavage site nomenclature, the residues of the substrate (target or target sequence) on the N-terminal side of the scissile bond are denoted as P1, P2, P3, P4, etc., in the C-to-N direction. On the C-terminal side of the scissile bond, the residues of the substrate are denoted P1′, P2′, P3′, P4′, etc., in the N-to-C direction.

A contemplated recombinant protease precursor can consequently be described as containing at least two polypeptide sequence portions, and a linking target sequence and scissile bond-containing portion between them.

A contemplated protease precursor can be free of glycosylation or can be glycosylated. Glycosylation-free proteins are readily expressed from bacteria, whereas mammalian cells typically express glycosylated proteins.

A first polypeptide sequence portion of that precursor contains an amino acid sequence of an enzymatically active protease whose optimal pH value of proteolytic activity is known. As discussed below, a few of the amino acid residues from the target sequence are typically present in the first polypeptide sequence portion and one or more of those residues can be heterologous to the native or wild type protease.

A second polypeptide sequence portion constitutes the activation peptide and contains the remainder of the target sequence that links the two polypeptide portions. The target sequence contains at least one heterologous amino acid residue and the scissile bond that is cleaved by the enzymatically active recombinant protease of the first sequence. The target polypeptide portion is peptide bonded to both polypeptide portions such that an active protease is provided when the target sequence scissile bond is cleaved by a first polypeptide portion.

Cleavage of the target polypeptide sequence occurs when the recombinant molecule is present in an aqueous buffer at the optimal pH value. Typically, the recombinant enzyme also cleaves the target sequence at pH values 1 to 2 units on either side of the optimal value. Thus, when dissolved or dispersed in an aqueous buffer at the optimal pH value for the protease, the first polypeptide sequence portion cleaves the target amino acid residue sequence in the absence of other enzymes.

A target amino acid residue sequence can contain two to about eight amino acid residues and is determined by the activity of the first polypeptide active site. For example, trypsin, a serine protease, cleaves the bond on the carboxyl side of a lysine or arginine bonded to any residue other than proline, thereby constituting a two residue target sequence.

Although a target sequence of a contemplated precursor molecule polypeptide sequence can contain up to eight heterologous amino acid residues, a target sequence need contain only one such residue. Typically, one to about six heterologous residues are present. When only one heterologous amino acid residue is present in the target sequence, that residue can remain a part of the first or the second polypeptide portion of a novel enzyme after cleavage of the scissile bond.

It is to be understood that the second polypeptide portion and the linking heterologous target sequence can be present peptide-bonded at one or more termini of the first polypeptide. It is also to be understood that the first polypeptide sequence portion can be a single polypeptide sequence, or can be a plurality such sequences that are cysteine-bonded (disulfide-bonded) together. Precursors of thrombin and activated protein C described herein are illustrative of polypeptide precursor molecules that contain one or more polypeptide sequences that are bonded together by cysteine disulfide bonds.

In some embodiments, the second polypeptide and linking target sequence are peptide-bonded at a terminus of the first polypeptide. In other embodiments, the second polypeptide sequence is peptide-bonded within the first polypeptide sequence.

When the second polypeptide sequence is peptide-bonded within the first polypeptide sequence, that second polypeptide sequence contains a plurality of linking target sequences that are cleaved by the first polypeptide portion. Each of those linking sequences can contain at least one heterologous amino acid residue. When two or more polypeptide cleavages are required to transform the precursor into an active protease, one or more additional linking polypeptide sequences can also be present that contain one or more other target sequences that is (are) cleaved by another one or more protease molecules.

For example, Chang, (1985) Eur. J. Biochem. 151:217-224 reported two cleavage sites sequences for thrombin that provided optimal cleavage rates. One contained hydrophobic residues at the P3 and P4 positions, followed by Pro-Arg-P1′-P2′, where P1′ and P2′ are nonacidic residues, whereas the second was P2-Arg-P1′, where P2 and P1′ are both Gly.

It is to be further understood that a scissile bond-containing target sequence linking polypeptide portion has a known, predetermined sequence for each recombinant protease precursor contemplated. Those target sequences and the preferences for particular amino acid residues at particular positions on either side of the scissile bond for separate proteases are readily found in the literature, and can be easily determined for any newly found protease.

As noted previously, a contemplated recombinant protease precursor contains at least 95 percent of the amino acid residue sequence of an active protease, including the active site. That number is calculated by inclusion of the one to about ten heterologous residues introduced in a target sequence or other polypeptide portion, which can be conservative or non-conservative substitutions. Heterologous amino acid residues present in a third polypeptide sequence portion as discussed below are not included in this percentage calculation. It is more preferred that the recombinant protease precursor contains at least 97 percent of the amino acid residue sequence of an active protease, and most preferred to contain at least 98 percent of the active protease sequence.

In some embodiments a heterologous residue be a conservative substitution for a residue of the wild type (native) protein. In other embodiments, a heterologous residue can be a non-conservative substitution. In some embodiments, both types of substitutions can be present. A worker skilled in biochemistry can readily determine whether a substitution is conservative or non-conservative.

For example, as illustrated hereinafter, replacement of three residues with acidic side-chains by alanines with small, hydrophobic side-chains are deemed to be three non-conservative substitutions. Similarly, the replacement of a glycine residue with a proline in the target sequence is also deemed to be a non-conservative substitution.

Where the activity of the protease is desired to be altered from that of the wild type enzyme to the auto-activation aspect, non-conservative substitutions are preferred, although such substitutions are often separated from an auto-activation cleavage site. An example of this alteration of the latter difference in enzymatic activity is found in the wild type and mutant or variant thrombins whose values of k_{cat}/Kₘ with various substrates are illustrated in Table 1 hereinafter. It is seen in that table that the values for the wild type (wt) and the auto-activating, non-conservatively substituted (EDE) thrombins having usual thrombin activity are within about a factor of 2-3, whereas the value for the auto-activating WE (EDEWE) thrombin is several orders of magnitude different from the wild type enzyme in usual thrombin activity.

In some preferred embodiments, one or more heterologous amino acid residues present in the sequence of a contemplated auto-lytic protease precursor alter the stereochemical conformation of the cleavage site to cause the activating bond-containing residue of the target sequence such as an Arg or Lys to extend into the aqueous solvent medium where that peptide bond can be cleaved, rather than being buried within the folded protein and protected from cleavage. The data discussed in Pozzi et al., (2011) Biochemistry 50(47):10195-10202 from crystallographic and proteolysis studies of substitution mutants of prothrombin-2 illustrate the effects of substituting three native acidic side-chained residues near the N-terminus of the molecule with neutral side-chained residues (E14eA/D141A/E18A). That set of substitutions was shown to lead to a change in the 3-dimensional position of the activation cleavage site (target sequence) that had been sterically blocked in the native molecule into being in contact with the solvent, and led to auto-lysis.

Thus, in such situations, the activation cleavage site (target sequence) of a native precursor has a sequence that can be cleaved by the enzyme of the precursor, but is not so cleaved because the cleavage site is sterically hindered. In that circumstance, a contemplated embodiment contains a target sequence that is a native sequence, and there are one to about six heterologous residues present in the precursor sequence that cause a change in the conformation of the target sequence cleavage site such that that sequence can be cleaved by a contemplated enzyme precursor.

Positioning of the location of the heterologous residue(s) can be determined by examination of the X-ray or other (e.g., NMR or electron cryomicroscopy) 3-dimensional structural analysis. The identity of the residues that are substituted and those utilized for substitution typically depends on whether a region is desired to flex inwardly or outwardly into the solvent. These protein chain flexions can be attained by adjusting hydrophobic/hydrophilic and/or electrostatic interactions as is well-known to a biochemist.

In some other preferred embodiments, the amino acid residue sequence of the activation cleavage site is altered by substitution of one to about six heterologous residues to provide an activation cleavage site split by the enzyme when such a site is not present in the native zymogen. Additionally, the one to about six heterologous residues can be utilized to provide a target site that is more readily cleaved by the enzyme than is a site present in the native sequence.

An example of such a target sequence substitution is illustrated hereinafter where a glycine at position G14m of native prothrombin-2 was substituted with a proline (i.e., IDGRIV vs IDPRIV) [SEQ ID NO:9 and SEQ ID NO:10]. The presence of a proline at P2 position in this target sequence made this sequence an ideal substrate for thrombin and therefore mutants G14mP and G14mP/EDE auto activate faster than the prototype mutant EDE.

An active recombinant protease enzyme that is the product of auto-activation of an above-described recombinant precursor can, but need not, contain one or more heterologous amino acid residues that are the residue of an engineered target sequence that has been cleaved by the active enzyme during auto-activation. An active enzyme that contains one or more such heterologous amino acid residues is also contemplated by this invention.

A contemplated protease precursor can also contain a third polypeptide sequence portion peptide-bonded at one or both termini that is useful during expression and/or purification and is subsequently cleaved from a precursor or the protease molecule. Such sequences are most usually at the N-terminus. Illustrative of such polypeptides are the 24 residue signal peptide at the N-terminus of a thrombin precursor and the N-terminal 32 amino acid residue signal peptide of activated protein C. An exemplary N-terminal third polypeptide portion can be a commonly expressed purification-assisting polypeptide such as FLAG peptide, P-galactosidase (P-Gal or LacZ), glutathione-S-transferase (GST) protein, a hexa-his peptide (6×His-tag), chitin binding protein (CBP), maltose binding protein (MBP), V5-tag, c-myc-tag, HA-tag, and the like as are well known.

One particularly preferred embodiment contemplates a serine protease precursor that contains at least two polypeptide portions as discussed above. That first polypeptide sequence is peptide-bonded to a second polypeptide sequence portion via a linking target amino acid residue sequence that contains the scissile bond that is cleaved by the enzymatically active serine protease. When dispersed in an aqueous buffer at the optimal pH value for the protease, the first polypeptide sequence portion cleaves the target amino acid residue sequence portion in the absence of other enzymes. The residuum of the second polypeptide portion and the target sequence are thereby typically separated from the remainder of the active protease.

Although a contemplated recombinant precursor can be expressed in an eukaryotic cell such as a mammalian or yeast cell, a contemplated recombinant protease precursor is often preferably expressed in a prokaryotic cell, and particularly a bacterial cell. A contemplated recombinant precursor protease is preferably bacteria-derived (-grown or -expressed), and is more preferably Escherichia coli culture-derived (or -expressed; E. coli-derived; E. coli-expressed). Bacterially-expressed, glycosylation-free protease precursors are also contemplated that contain the amino acid residue sequences of active proteases.

Thus, a particular aspect of the present invention contemplates a bacteria-derived (-grown or -expressed) recombinant thrombin precursor that contains the amino acid residue sequence of mutant thrombin EDE, listed in SEQ ID NO:1, or mutant thrombin EDEWE, listed in SEQ ID NO:2, and is preferably Escherichia coli culture-derived (or -expressed; E. coli-derived; E. coli-expressed). A bacterially-expressed, glycosylation-free thrombin precursor is also contemplated that contains the SEQ ID NO:1 or SEQ ID NO:2 amino acid residue sequence.

In another aspect of the invention, a protease precursor polypeptide containing an amino acid residue sequence whose thrombin portion (the portion that forms thrombin) is at least about 95 percent identical to the amino acid residue sequence of wild type human thrombin of SEQ ID NO:3. More preferably, a thrombin portion is about 97 percent or more identical to that of wild type human thrombin of SEQ ID NO:3, most preferably, the identity is about 98 percent or more.

Another aspect of the present invention contemplates a mammalian-derived (-grown or -expressed) recombinant activated protein C precursor that contains the SEQ ID NO:8 amino acid residue sequence and is preferably BHK culture-derived (or -expressed). A mammalian-expressed, activated protein C precursor is also contemplated that contains the SEQ ID NO:8 amino acid residue sequence.

In a further aspect of the invention, a protease precursor polypeptide containing an amino acid residue sequence whose activated protein C portion (the portion that forms activated protein C) is at least about 95 percent identical to the amino acid residue sequence of wild type human activated protein C of SEQ ID NO:6. More preferably, a protein C portion is about 97 percent or more identical to that of wild type human activated protein C of SEQ ID NO:6, most preferably, the identity is about 98 percent or more. This embodiment would include the mutant activated protein C amino acid residue sequence listed in SEQ ID NO:8.

It is also to be noted that a contemplated protease precursor need not be a well-known protease or protease precursor as discussed above. A protease precursor can be chosen from any protease for which the following are known: 1) the amino acid sequence for the enzymatically active portion of the protease; 2) the amino acid sequence(s) cleaved by the active protease; and 3) the optimal pH value of proteolytic activity.

A contemplated protease precursor can be viewed as an expressible fusion protein (polypeptide) in which the N-terminal portion of the fusion polypeptide provides a convenient sequence for expression and/or purification (expression/purification), whose C-terminal residue is peptide-bonded to a protease precursor sequence as discussed above. Thus, the N terminal portion of the expressed fusion polypeptide (protein) is a convenient expression/purification sequence, whereas the C-terminal portion has a desired protease precursor sequence, and the two portions are joined (linked) by the amino acid residue sequence of the target cleavage site of the protease.

An exemplary N-terminal fusion polypeptide portion can be a commonly expressed polypeptide such as FLAG peptide, .beta.-galactosidase (β-Gal or LacZ), glutathione-S-transferase (GST) protein, a hexa-his peptide (6×His-tag), chitin binding protein (CBP), maltose binding protein (MBP), V5-tag, c-myc-tag, HA-tag, and the like as are well known. The carboxy-terminus of the N-terminal fusion polypeptide portion is peptide bonded to a protease cleavage site as discussed above and that cleavage sequence is peptide-bonded to the incipient N-terminal residue of a desired protease precursor sequence that constitutes the carboxy-terminal portion of the fusion protein or polypeptide.

Alternatively, a contemplated protease precursor can be viewed as an expressible fusion protein (polypeptide) in which the C-terminal portion of the fusion polypeptide provides a convenient sequence for expression and/or purification (expression/purification), whose N-terminal residue is peptide-bonded to a protease precursor sequence as discussed above.

The present invention enables large-scale production of recombinant protease precursors, such as recombinant serine protease precursors like thrombin precursors, recombinant activated protein C precursors, and recombinant chymotrypsin precursors and the like for in vitro and in vivo studies, therapies, and other applications that are discussed herein.

A contemplated protease precursor expressed in bacteria (e.g., E. coli) is free of glycosylation and can be used therapeutically. One illustrative example includes the production of thrombin protease precursor for enhancing hemostasis or treating and preventing thrombosis.

One advantage of the present invention is that it permits faster and more economical production of large quantities of active proteases. In particular, bacteria such as E. coli can be used to produce large batches of active thrombin (or other active protease) for pharmaceutical development, therapy and other uses.

### Compositions and Methods

Methods for making the proteins and nucleotides used in the invention, as well as the methods of the invention taught in this disclosure utilize the conventional techniques of molecular genetics, cell biology, and biochemistry. Useful methods in molecular genetics, cell biology and biochemistry are described in Molecular Cloning: A Laboratory Manual, 2nd Ed. (Sambrook et al., 1989); Oligonucleotide Synthesis (M. J. Gait, ed., 1984); Animal Cell Culture (R. I. Freshney, ed., 1987); the series Methods in Enzymology (Academic Press, Inc.); “Gene Transfer Vectors for Mammalian Cells” (J. M. Miller & M. P. Calos, eds., 1987); Current Protocols in Molecular Biology and Short Protocols in Molecular Biology, 3rd Edition (F. M. Ausubel et al., eds., 1987 & 1995); and Recombinant DNA Methodology II (R. Wu ed., Academic Press 1995). Methods for peptide synthesis and manipulation are described in Solid Phase Peptide Synthesis, (J. M. Stewart & J. D. Young, 1984); Solid Phase Peptide Synthesis: A Practical Approach (E. Atherton & R. C. Sheppard, 1989); The Chemical Synthesis of Peptides (J. Jones, International Series of Monographs on Chemistry vol. 23, 1991); and Solid Phase Peptide Synthesis, (G. Barany & R. B. Merrifield, Chapter 1 of The Peptides, 1979); and Bioconjugate Techniques (G. T. Hermanson, 1996).

In some embodiments, a contemplated protease precursor is expressed in eukaryotic host cells. The protease precursor polypeptide so expressed is glycosylated. Illustrative eukaryotic cells include insect cells such as Sf9, and mammalian cell lines such as CHO, COS, 293, 293-EBNA, BHK, HeLa, NIH/3T3, and the like. Exemplary yeast host cells include Saccharomyces cerevisiae, Pichia pastoris, Hansenula polymorpha, Kluyveromyces lactis, Schwanniomyces occidentis, Schizosaccharomyces pombe and Yarrowia lipolytica.

More preferably, a contemplated protease precursor polypeptide is expressed in prokaryotic cells. Preferred prokaryotic cells are bacteria cells. Preferred bacteria cells are E. coli cells. Several strains of Salmonella such as S. typhi and S. typhimurium and S. typhimurium-E. coli hybrids can also be used to express a contemplated protease precursor. See, U.S. Pat. No. 6,024,961; U.S. Pat. No. 5,888,799; U.S. Pat. No. 5,387,744; U.S. Pat. No. 5,297,441; Ulrich et al., (1998) Adv. Virus Res., 50:141-182; Tacket et al., (1997) Infect. Immun., 65(8):3381-3385; Schodel et al., (1997) Behring Inst. Mitt., 98:114-119; Nardelli-Haefliger et al., (1996) Infect. Immun., 64(12):5219-5224; Londono et al., (1996) Vaccine, 14(6):545-552, and the citations therein.

A preferred E. coli strain useful herein for expression of a contemplated protease precursor is BL21 (DE3). Additional E. coli strains useful for expression include XL-1, TB1, JM103, BLR, pUC8, pUC9, and pBR329 (Biorad Laboratories, Richmond, Calif.) and pPL and pKK223-3 available from (Pharmacia, Piscataway, N.J.).

A bacterial host that expresses a contemplated recombinant protease precursor is a prokaryote, such as E. coli, and a preferred vector includes a prokaryotic replicon; i.e., a DNA sequence having the ability to direct autonomous replication and maintenance of the recombinant DNA molecule extrachromosomally in a prokaryotic host cell transformed therewith. Such replicons are well known in the art. Vectors that include a prokaryotic replicon can also include a prokaryotic promoter region capable of directing the expression of a protease precursor gene in a host cell, such as E. coli, transformed therewith.

Promoter sequences compatible with bacterial hosts are typically provided in plasmid vectors containing one or more convenient restriction sites for insertion of a contemplated DNA segment. Illustratively useful promoters and vectors include the Rec 7 promoter that is inducible by exogenously supplied nalidixic acid. A more preferred promoter is present in plasmid vector JHEX25 (Promega, Madison, Wis.) that is inducible by exogenously supplied isopropyl-.beta.-D-thiogalacto-pyranoside (IPTG). Another preferred promoter, the tac (a hybrid of the trp and lac promoter/operator), is present in plasmid vector pKK223-3 (Pharmacia, Piscataway, N.J.) and is also inducible by exogenously supplied IPTG. Further promoters and promoter/operators include the araB, trp, lac, gal, T7, and the like are useful in accordance with the instant invention.

The exact details of the expression construct vary according to the particular host cell that is to be used as well as to the desired characteristics of the expression system, as is well known in the art. For example, for production in S. cerevisiae, the DNA encoding a thrombin precursor of the invention is placed into operable linkage with a promoter that is operable in S. cerevisiae and which has the desired characteristics (e.g., inducible/derepressible or constitutive), such as GAL1-10, PHOS5, PGK1, GDP1, PMA1, MET3, CUP1, GAP, TPI, MFα1 and MFα2, as well as the hybrid promoters PGK/α2, TPI/α2, GAP/GAL, PGK/GAL, GAP/ADH2, GAP/PHO5, ADH2/PHO5, CYC1/GRE, and PGK/ARE and other promoters known in the art.

When other eukaryotic cells are the desired host cell, any promoter active in the host cell may be utilized. For example, when the desired host cell is a mammalian cell line, the promoter can be a viral promoter/enhancer (e.g., the herpes virus thymidine kinase (TK) promoter or a simian virus promoter (e.g., the SV40 early or late promoter) or the Adenovirus major late promoter, a long terminal repeat (LTR), such as the LTR from cytomegalovirus-(CMV), Rous sarcoma virus (RSV) or mouse mammary tumor virus (MMTV)) or a mammalian promoter, preferably an inducible promoter such as the metallothionein or glucocorticoid receptor promoters and the like.

Expression constructs can also include other DNA sequences appropriate for the intended host cell. For example, expression constructs for use in higher eukaryotic cell lines (e.g., vertebrate and insect cell lines) include a poly-adenylation site and can include an intron (including signals for processing the intron), as the presence of an intron appears to increase mRNA export from the nucleus in many systems. Additionally, a secretion signal sequence operable in the host cell is normally included as part of the construct. The secretion signal sequence for a thrombin precursor can, for example, be the naturally occurring preprothrombin signal sequence, or it can be derived from another gene, such as human serum albumin, human prothrombin, human tissue plasminogen activator, or preproinsulin. Where the expression construct is intended for use in a prokaryotic cell, the expression construct can include a signal sequence that directs transport of the synthesized polypeptide into the periplasmic space or expression can be directed intracellularly.

Preferably, the expression construct also comprises a means for selecting for host cells that contain the expression construct (a “selectable marker”). Selectable markers are well known in the art. For example, the selectable marker can be a resistance gene, such as aN antibiotic resistance gene (e.g., the neo^{r} gene that confers resistance to the antibiotic gentamycin or the hyg^{r} gene that confers resistance to the antibiotic hygromycin). Alternatively, the selectable marker can be a gene that complements an auxotrophy of the host cell. If the host cell is a Chinese hamster ovary (CHO) cell that lacks the dihydrofolate reductase (dhfr) gene, for example CHO DUXB11 cells, a complementing dhfr gene would be preferred.

If the host cell is a yeast cell, the selectable marker is preferably a gene that complements an auxotrophy of the cell (for example, complementing genes useful in S. cerevisiae, P. pastoris and S. pombe include LEU2, TRP1, TRP1d, URA3, URA3d, HIS3, HIS4, ARG4, LEU2d), although antibiotic resistance markers such as SH BLE, which confers resistance to ZEOCIN®, can also be used. If the host cell is a prokaryotic or higher eukaryotic cell, the selectable marker is preferably an antibiotic resistance marker (e.g., neo^{r}). Alternately, a separate selectable marker gene is not included in the expression vector, and the host cells are screened for the expression of a thrombin precursor (e.g., upon induction or derepression for controllable promoters, or after transfection for a constitutive promoter, fluorescence-activated cell sorting, FACS, may be used to select those cells which express the recombinant thrombin precursor). Preferably, the expression construct comprises a separate selectable marker gene.

A suitable promoter or enhancer, termination sequence and other functionalities for use in the expression of a protease precursor in given recombinant host cells are well known, as are suitable host cells for transfection with nucleic acid encoding the desired variant proteases. It can be useful to use host cells that are capable of glycosylating the variant protease precursors, which typically include mammalian cells as discussed before.

In addition, host cells are suitable that have been used heretofore to express proteolytic enzymes or zymogens in recombinant cell culture, or which are known to already express high levels of such enzymes or zymogens in non-recombinant culture. In the latter case, if the endogenous enzyme or protease precursor is difficult to separate from a variant protease precursor, the endogenous gene should be removed by homologous recombination or its expression suppressed by cotransfecting the host cell with nucleic acid encoding an anti-sense sequence that is complementary to the RNA encoding the undesired polypeptide. In this case, the expression control sequences (e.g., promoter, enhancers, etc.) used by the endogenous expressed gene optimally are used to control expression of a protease precursor variant.

A method of preparing a serine protease as described above and elsewhere herein is also contemplated. In accord with that method, a recombinant serine protease precursor as discussed above and elsewhere is dissolved or dispersed in an aqueous buffer to form a composition, with the aqueous buffer being at a pH value suitable for cleavage by the protease. A suitable pH value includes the optimal pH value for the protease, as well as pH values that are typically about two units on either side of the optimal pH value for cleavage. For example, activated protein C is reported to have an optimal pH value for activity about 8.5 under the conditions studied. [Ohno et al., (1981) J Biochem 90(5):1387-395.]

The composition is maintained for a time sufficient for the recombinant serine protease precursor to cleave itself and form the recombinant serine protease. The maintenance time can be from an hour to a few days.

The aqueous buffer is preferably maintained at about room temperature, but can be at any temperature at which neither the precursor nor the enzyme itself is degraded. These values are typically found in the literature and can be readily obtained by a skilled worker using standard techniques. For example, thrombin was reported to have an optimum activity for the cleavage of Tos-Gly-Pro-Arg-pNa to release p-nitroaniline at 45° C. [Le Borgne et al., (1994) Appl. Biochem Biotech 48:125-135.] Usual temperatures are about zero ° C. to about 50° C., and more preferably about 20° C. to about 40° C. The protease so prepared can be used without further isolation and purification, or can be recovered and purified to a desired extent.

The following examples are for illustrative purposes and are in no way limiting.

### Example 1

### Protocol for E. coli Expression of Thrombin Mutants EDE and EDEWE

The cDNA corresponding to the prethrombin-2 sequence of human thrombin was cloned into the pET21a vector (Novagen) using the EcoRI and the XhoI restriction sites. Site-directed mutagenesis was performed using the QUIKCHANGE® site-directed mutagenesis kit from Stratagene (La Jolla, Calif.).

The prethrombin-2 vector was transformed into BL21(DE3). E. coli cells grown overnight (about 18 hours) in 50 ml of LB medium with 0.1 mg/ml ampicillin at 37° C. and 225 rpm. The next morning, 3 liters of LB medium with 0.1 mg/ml of ampicillin was inoculated with 50 ml of over-night (about 18 hours) culture. Growth was continued at 37° C. and 225 rpm until the cells reached A₆₀₀=0.6.

Prethrombin-2 expression was initiated by adding IPTG to a final concentration of 1 mM. E. coli cells were then cultured for an additional 6 hours and cultures were spun at 4000 rpm for 15 minutes at 4° C. The cell paste was stored at −80° C.

The cell paste was thawed at 37° C. and resuspended in 75 ml of 50 mM Tris, pH 7.4 at 25° C., 20 mM EDTA, 0.1% TRITON® X-100, 20 mM DTT. Cells were further sonicated on ice for 5 cycles of 30 seconds of sonication in between 1-minute rest periods. The well-homogenized cells were ultracentrifuged for 20 minutes at 4° C., 10,000×g. The supernatant was discarded, and the pellet was resuspended in 75 ml of 50 mM Tris, pH 7.4 at 25° C., 20 mM EDTA, 0.1% TRITON® X-100.

The homogenate was centrifuged for 20 minutes at 10,000×g at 4° C. Supernatant was discarded and the pellet was suspended in 75 ml of 50 mM Tris, pH 7.4 at 25° C., 20 mM EDTA, 1 M NaCl prior to centrifugation for 20 minutes at 10,000×g at 4° C. This step was repeated 2 additional times, until the pellet became white. The supernatant was then discarded, and the pellet was resuspended in 75 ml of 50 mM Tris, pH 7.4 at 25° C., 20 mM EDTA, The suspension was finally spun at 10,000×g for 30 minutes at 4° C.

Inclusion bodies from 1 L of cells were solubilized via addition of 7 M Gnd-HCl and 30 mM L-cysteine to a final concentration of 30-40 mg/mL.

After 2-3 hours at room temperature, the unfolded protein was first diluted into 6 M Gnd-HCl, 0.6 M L-arginine HCl, 50 mM Tris (pH 8.3), 0.5 M NaCl, 1 mM EDTA, 10% glycerol, 0.2% BRIJ® 58, and 1 mM L-cysteine, then refolded by reverse dilution to a final concentration of 0.15-0.2 mg/mL and finally maintained for 6-10 hours at room temperature.

The refolded protein in 0.6 M L-arginine HCl, 50 mM Tris (pH 8.3), 0.5 M NaCl, 1 mM EDTA, 10% glycerol, 0.2% BRIJ® 58, and 1 mM L-cysteine was extensively dialyzed against 10 mM Tris (pH 7.4), 0.2 M NaCl, 2 mM EDTA, and 0.1% polyethylene glycol (PEG) 6000 for 24-30 hours at room temperature and then, after centrifugation and filtration, loaded overnight (about 18 hours) onto a 5 mL heparin-Sepharose column.

Alternatively the refolded protein was concentrated from 1 liter to 150 ml using Quickstand™ filtration system, from Amersham Biosciences, with a 10-kDa hollow fiber cartridge. The 150 ml of refolded protein was dialyzed against three changes of 20 mM Tris, pH 7.0 at 25° C., 0.15 M NaCl. Precipitate was removed by centrifugation.

Protein solution was loaded onto a 5-ml heparin column (GE Healthcare), at 2.5 ml/minute. The bound protein was extensively washed with 20 mM Tris 200 mM NaCl, pH 7.4 at 25° C. before elution with a linear gradient to 2 M NaCl. The elution was monitored by UV spectroscopy and the fractions containing the UV peak were collected.

For the wild type protein, activation was carried out using 10 μl of ecarin (50 EU/ml) per 1 ml of protein solution. Activation was monitored from the hydrolysis of the chromogenic substrate H-D-Phe-Pro-Arg-p-nitroanilide (FPR). The activated protein was diluted 4-fold and purified as described before.

Values of s=k_{cat}/Kₘ for release of fibrinopeptide A (FpA) from fibrinogen, cleavage of the protease-activated receptor PAR1 and activation of protein C in the presence of 50 nM thrombomodulin and 5 mM CaCl₂ were obtained as reported elsewhere [Di Cera, (2008) Mol Aspects Med 29:203-254; Chen et al., (2010) Proc Natl Acad Sci USA 107:19278-19283; and Marino e al., J. Biol. Chem. 285:19145-19152] under experimental conditions of 5 mM Tris, 0.1% PEG8000, 145 mM NaCl, pH 7.4 at 37° C.

The data are found in Table 1 below. The conclusion reached is that mutants thrombin EDE and thrombin EDEWE cleave synthetic and physiological substrates with values of k_{cat}/kₘ comparable to those of wild type thrombin.

TABLE 1
Values of k_{cat}/Kₘ (μM⁻¹s⁻¹) for wild type and mutant thrombins toward synthetic and
physiological substrates

| Enzyme | FPR | FpA | PAR1 | Protein C |
|---|---|---|---|---|
| Wt | 37 ± 1 | 17 ± 1 | 27 ± 1 | 0.22 ± 0.01 |
| EDE | 19 ± 1 | 7.2 ± 0.5 | 9.1 ± 0.6 | 0.11 ± 0.01 |
| EDEWE | 0.0015 ± 0.0001 | 0.00026 ± 0.00001 | 0.016 ± 0.001 | 0.031 ± 0.001 |

### Example 2

### E. Coli-Expressed Protease Precursors Thrombin EDE and Thrombin-EDEWE Auto-Activate Themselves

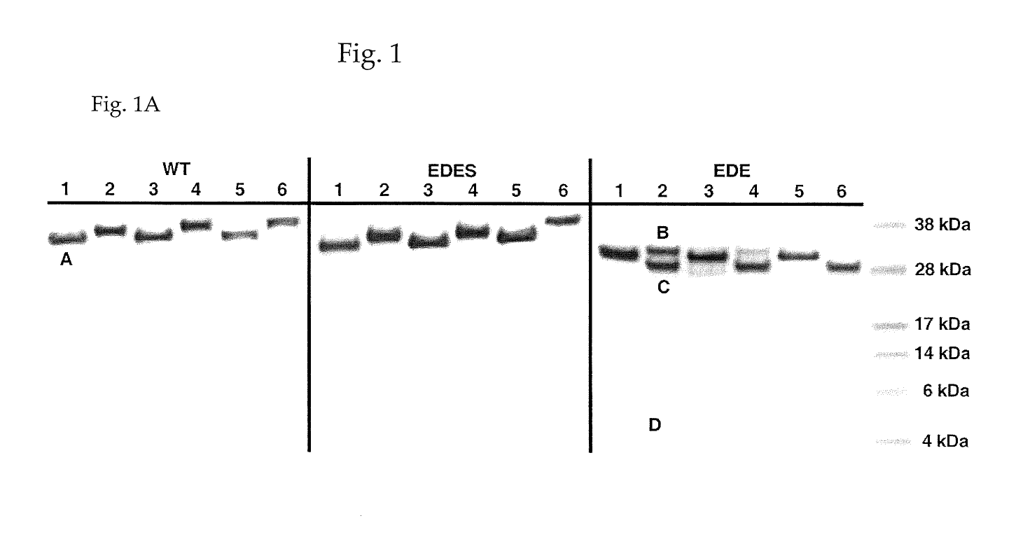

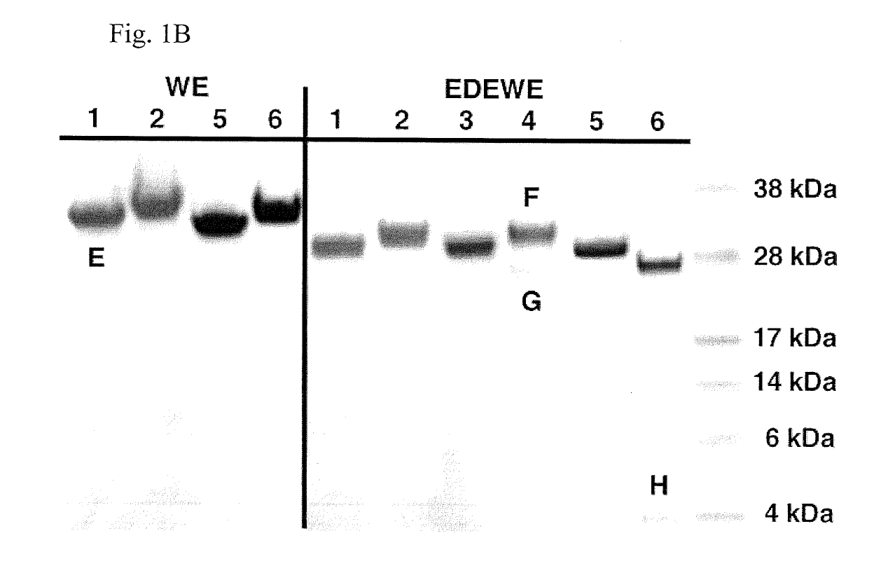

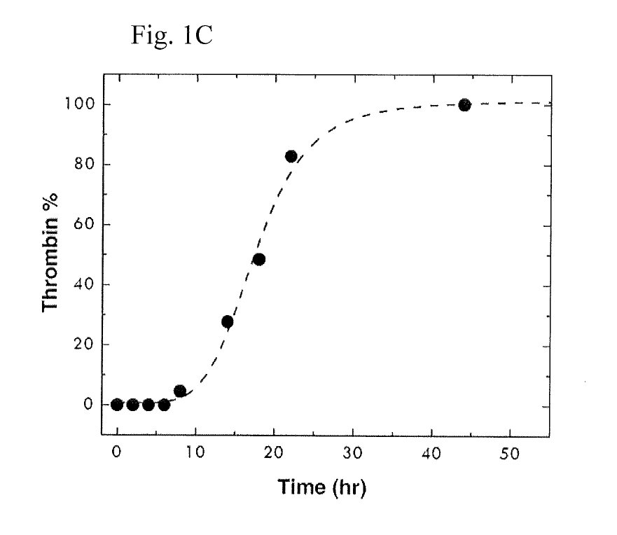

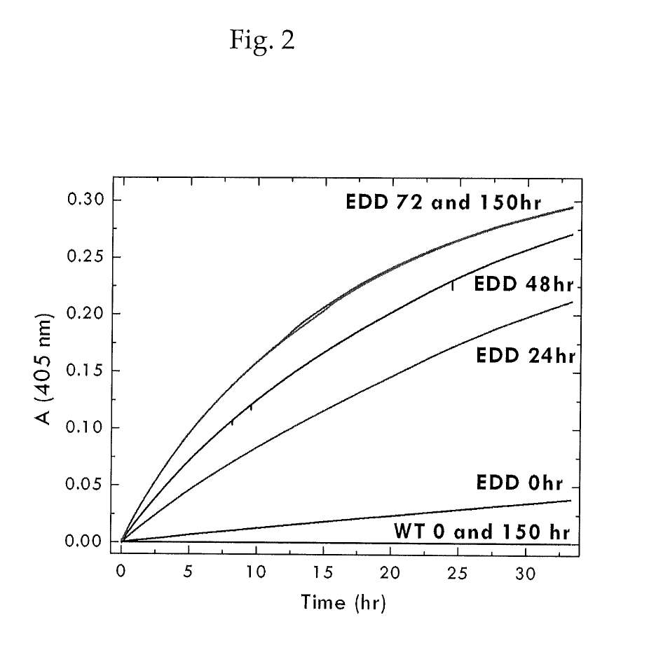

The ability of recombinant protease to activate itself is shown in FIG. 1. After heparin-sepharose purification, the concentration of each protein was adjusted to 1.1 mg/ml and auto-activation was followed at room temperature for 0 (A, lanes 1, 2), 4 (A, lanes 3, 4) and 90 (A, lanes 5, 6) hours.

Auto-activation is also observed when the E14eA/D141A/E18A mutation is introduced in the prethrombin-2 mutant W215A/E217A (WE) to yield the construct E14eA/D141A/E18A/W215A/E217A (EDEWE). In this case, the concentration was adjusted to 3 mg/ml and the reaction was followed at room temperature for 0 (B, lanes 1, 2), 3 (B, lanes 3, 4) and 7 (B, lanes 5, 6) days. No evidence of auto-activation was detected for WE over the same time scale.

Samples were analyzed under non-reducing (B, lanes 1, 3, 5) and reducing (B, lanes 2, 4, 6) conditions. In the case of EDE and EDEWE, the two bands pertaining to the A and B chains of the mature enzyme are easily detected under reducing conditions and conversion to thrombin is complete after 90 hours or 7 days, respectively.

The chemical identity of the A and B chains was confirmed by N-terminal sequencing. Bands in the gel are labeled as follows: A and E mapped to N-terminal sequence GRGSE and refer to prethrombin-2 constructs with the T7tag from the expression vector partially cleaved and then processed during E. coli expression; B and F mapped to N-terminal sequence TFGSG and refer to prethrombin-2 with a single N-terminus starting at Tlh; C and G mapped to N-terminal sequence IVAGS and refer to the B chain of thrombin with the N-terminus 116 and the mutation E18A introduced in the EDE and EDEWE constructs; D and H mapped to N-terminal sequence TFGSG and refer to the A chain of thrombin with the N-terminus Tlh.

Kinetics of auto-activation of prethrombin-2 EDE was monitored as percent of thrombin created. The shape of the auto-activation curve is consistent with an autocatalytic process initiated by prethrombin-2 EDE itself and leading to complete conversion to thrombin (C).

### Example 3

### Expression of Prethrombin-1 Mutant EDE

Site-directed mutagenesis of human thrombin was carried out in a HPC4-modified pNUT expression vector by using the QUICK-CHANGE® Site-Directed Mutagenesis Kit from Stratagene.

After validation of the constructs, proteins were expressed in BHK cells in media containing DMEM supplemented with 10% (V/V) calf serum and 2 mM L-Glutamine. Right after collecting cell culture supernatant, benzamidine HCl was added to a 5 mM final concentration to prevent proteolysis.

Both wild-type and mutants were purified to homogeneity by immunoaffinity chromatography using the Ca²⁺-dependent monoclonal antibody HPC4. Eluted proteins were concentrated using VIVASPIN® concentrators (Sartorius Stedim Biotech, Germany) and loaded onto a gel filtration Superdex™ 200 column (GE Healtcare, Bio-Sciences AB, Uppsala, Sweden).

After the gel filtration step, protein concentration was adjusted to 1.1 mg/mL and autoactivation was followed at room temperature for up to 120 hours. To visualize autoactivation in polyacrylamide gels, time reaction aliquots were collected and quenched with reducing SDS protein loading buffer and immediately stored at −80° C. Samples were processed and analyzed by SDS-PAGE electrophoresis and gels were stained with Coomassie brilliant blue R250.

### Example 4

Expression of Activated Protein C Mutant

Preparation of vectors, protein expression and purification of Gla-domainless protein C wild-type and mutants E160A/D167A/D172A (EDD) and E160A/D167A/D172A/S 360A (EDDS) were carried out as described elsewhere [Rezaie et al., J Biol. Chem. 1992 267:26104-26109]. Primers used for the EDD mutant were (forward)

SEQ ID NO: 11
5′-CACAGCAGACCAAGAAGACCAAGTAGCTCCGCGGCTCATTGCTG
GG-3′
and
(reverse)
SEQ ID NO: 12
5′-CCCAGCAATGAGCCGCGGAGCTACTTGGTCTTCTTGGTCTGCTG
TG-3′.
For the EDDS mutant, the primers were (forward)
SEQ ID NO: 13
5′-TGCCTGCGAGGGCGACGCTGGGGGGCCCATGGTC-3′
and
(reverse)
SEQ ID NO: 14
5′-GACCATGGGCCCCCCAGCGTCGCCCTCGCAGGCA-3′.

After validation of the constructs, proteins were expressed in BHK cells in media containing DMEM supplemented with 10% (V/V) calf serum and 2 mM L-Glutamine. Right after collecting cell culture supernatant, benzamidine HCl was added to a 5 mM final concentration to prevent proteolysis.

Both wild-type and mutants were purified to homogeneity by immunoaffinity chromatography using the Ca²⁺-dependent monoclonal antibody HPC4. Eluted proteins were concentrated using VIVASPIN® concentrators (Sartorius Stedim Biotech, Germany) and loaded onto a gel filtration SUPERDEX™ 200 column (GE Healtcare, Bio-Sciences AB, Uppsala, Sweden).

After the gel filtration step, protein concentration was adjusted to 0.8 mg/mL and autoactivation was followed at room temperature for up to 150 hours. To visualize autoactivation in polyacrylamide gels, time reaction aliquots were collected and quenched with reducing SDS protein loading buffer and immediately stored at −80° C.

Samples were processed and analyzed by SDS-PAGE electrophoresis and gels were stained with Coomassie brilliant blue R250.

The kinetics of autoactivation were monitored by collecting samples over time and measuring activity toward the chromogenic substrate H-D-Asp-Arg-Arg-p-nitroanilide (DRR) specific for activated protein C [Dang et al., (1997) Blood 89(6):2220-2222] under experimental conditions of 10 mM Tris, pH 7.4, 145 mM NaCl, 2 mM CaCl₂, 0.1% PEG8000 at 37° C., and in the presence of 1 M hirudin as a control to rule out contaminating effects from thrombin.

### Example 5

### Control of Protease Precursor Activation Rate with Salt Concentrations and pH

Consistently with a thrombin-catalyzed mechanism, the rate of autoactivation can be efficiently modulated by monovalent cations, specifically Na⁺>K⁺>Ch⁺ (choline). In the presence of Na⁺ and at a final concentration of 1.1 mg/ml, 50% of the reaction is achieved after 17 hours, 2.5 and >10 times faster with respect to K⁺ and Ch⁺, respectively. Specific activation of thrombin by Na⁺ represents an important hallmark of this coagulation factor.

Changing buffers also modulates autoactivation, accordingly with the well-known effect of the pH on the catalytic activity of the serine proteases. Below pH 5.5, prethrombin-2 EDE appears to be stable over time, whereas the fastest activation rate is observed at pH 8.5. Possible contaminations by exogenous proteases were ruled out by blocking autoactivation using hirudin as a specific thrombin inhibitor, by adding 2 mM EDTA as potent chelator for bivalent cations and by introducing the mutation S195A in the triple mutant EDE. The resulting mutant EDES does not convert into thrombin spontaneously, demonstrating that the low but significant activity of the zymogen form is key to starting the autoactivation.

### Example 6

### Optimization of the Autoactivation Rate

To further optimize the rate of the autoactivation process, the glycine at position G14m was substituted with a proline (i.e. IDGRIV vs IDPRIV) [SEQ ID NO:9 and SEQ ID NO: 10]. The presence of a proline at P2 position made this sequence an ideal substrate for thrombin and therefore mutants G14mP and G14mP/EDE auto activate faster than the prototype mutant EDE.

This construct E14eA/D141A/G14mP/E18A/(EDGE) thus contains heterologous residues at in the target sequence (three-DGE) and elsewhere in the precursor sequence (one-E). A similar construct prepared from the (E14eA/D141A/E18A/W215A/E217A) EDEWE mutant of Example 2 would contain 6 heterologous residues E14eA/D141A/G14mP/E18A/W215A/E217A (EDGEWE).

Auto-lytic Thrombin (EDE)
SEQ ID NO: 1
TFGSGEADCG LRPLFEKKSL EDKTERALLE SYIAGRIVAG
SDAEIGMSPW QVMLFRKSPQ ELLCGASLIS DRWVLTAAHC
LLYPPWDKNF TENDLLVRIG KHSRTRYERN IEKISMLEKI
YIHPRYNWRE NLDRDIALMK LKKPVAFSDY IHPVCLPDRE
TAASLLQAGY KGRVTGWGNL KETWTANVGK GQPSVLQVVN
LPIVERPVCK DSTRIRITDN MFCAGYKPDE GKRGDACEGD
SGGPFVMKSP FNNRWYQMGI VSWGEGCDRD GKYGFYTHVF
RLKKWIQKVI DQFGE
Auto-lytic WE Thrombin
SEQ ID NO: 2
TFGSGEADCG LRPLFEKKSL EDKTERALLE SYIAGRIVAG
SDAEIGMSPW QVMLFRKSPQ ELLCGASLIS DRWVLTAAHC
LLYPPWDKNF TENDLLVRIG KHSRTRYERN IEKISMLEKI
YIHPRYNWRE NLDRDIALMKL KKPVAFSDY IHPVCLPDRE
TAASLLQAGY KGRVTGWGNL KETWTANVGK GQPSVLQVVN
LPIVERPVCK DSTRIRITDN MFCAGYKPDE GKRGDACEGD
SGGPFVMKSP FNNRWYQMGI VSAGAGCDRD GKYGFYTHVF
RLKKWIQKVI DQFGE
Thrombin
SEQ ID NO: 3
TFGSGEADCG LRPLFEKKSL EDKTERELLE SYIDGRIVEG
SDAEIGMSPW QVMLFRKSPQ ELLCGASLIS DRWVLTAAHC
LLYPPWDKNF TENDLLVRIG KHSRTRYERN IEKISMLEKI
YIHPRYNWRE NLDRDIALMK LKKPVAFSDY IHPVCLPDRE
TAASLLQAGY KGRVTGWGNL KETWTANVGK GQPSVLQVVN
LPIVERPVCK DSTRIRITDN MFCAGYKPDE GKRGDACEGD
SGGPFVMKSP FNNRWYQMGI VSWGEGCDRD GKYGFYTHVF
RLKKWIQKVI DQFGE
Prethrombin-2
SEQ ID NO: 4
TATSEYQTFF NPRTFGSGEA DCGLRPLFEK KSLEDKTERE
LLESYIDGRI VEGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKETWTAN
VGKGQPSVLQ VVNLPIVERP VCKDSTRIRI TDNMFCAGYK
PDEGKRGDAC EGDSGGPFVM KSPFNNRWYQ MGIVSWGEGC
DRDGKYGFYT HVFRLKKWIQ KVIDQFGE
Auto-lytic Prethrombin 2 (EDE)
SEQ ID NO: 5
TATSEYQTFF NPRTFGSGEA DCGLRPLFEK KSLEDKTERA
LLESYIAGRI VAGSDAEIGM SPWQVMLFRK SPQELLCGAS
LISDRWVLTA AHCLLYPPWD KNFTENDLLV RIGKHSRTRY
ERNIEKISML EKIYIHPRYN WRENLDRDIA LMKLKKPVAF
SDYIHPVCLP DRETAASLLQ AGYKGRVTGW GNLKETWTAN
VGKGQPSVLQ VVNLPIVERP VCKDSTRIRI TDNMFCAGYK
PDEGKRGDAC EGDSGGPFVM KSPFNNRWYQ MGIVSWGEGC
DRDGKYGFYT HVFRLKKWIQ KVIDQFGE
Preprotein C
SEQ ID NO: 6
MWQLTSLLLF VATWGISGTP APLDSVFSSS ERAHQVLRIR
KRANSFLEEL RHSSLERECI EEICDFEEAK EIFQNVDDTL
AFWSKHVDGD QCLVLPLEHP CASLCCGHGT CIDGIGSFSC
DCRSGWEGRF CQREVSFLNC SLDNGGCTHY CLEEVGWRRC
SCAPGYKLGD DLLQCHPAVK FPCGRPWKRM EKKRSHLKRD
TEDQEDQVDP RLIDGKMTRR GDSPWQVVLL DSKKKLACGA
VLIHPSWVLT AAHCMDESKK LLVRLGEYDL RRWEKWELDL
DIKEVFVHPN YSKSTTDNDI ALLHLAQPAT LSQTIVPICL
PDSGLAEREL NQAGQETLVT GWGYHSSREK EAKRNRTFVL
NFIKIPVVPH NECSEVMSNM VSENMLCAGI LGDRQDACEG
DSGGPMVASF HGTWFLVGLV SWGEGCGLLH NYGVYTKVSR
YLDWIHGHIR DKEAPQKSWA P
Protein C Zymogen
SEQ ID NO: 7
ANSFLEELRH SSLERECIEE ICDFEEAKEI FQNVDDTLAF
WSKHVDGDQC LVLPLEHPCA SLCCGHGTCI DGIGSFSCDC
RSGWEGRFCQ REVSFLNCSL DNGGCTHYCL EEVGWRRCSC
APGYKLGDDL LQCHPAVKFP CGRPWKRMEK KRSHLKRDTE
DQEDQVDPRL IDGKMTRRGD SPWQVVLLDS KKKLACGAVL
IHPSWVLTAA HCMDESKKLL VRLGEYDLRR WEKWELDLDI
KEVFVHPNYS KSTTDNDIAL LHLAQPATLS QTIVPICLPD
SGLAERELNQ AGQETLVTGW GYHSSREKEA KRNRTFVLNF
IKIPVVPHNE CSEVMSNMVS ENMLCAGILG DRQDACEGDS
GGPMVASFHG TWFLVGLVSW GEGCGLLHNY GVYTKVSRYL
DWIHGHIRDK EAPQKSWAP
Auto-lytic Protein C Zymogen
SEQ ID NO: 8
ANSFLEELRH SSLERECIEE ICDFEEAKEI FQNVDDTLAF
WSKHVDGDQC LVLPLEHPCA SLCCGHGTCI DGIGSFSCDC
RSGWEGRFCQ REVSFLNCSL DNGGCTHYCL EEVGWRRCSC
APGYKLGDDL LQCHPAVKFP CGRPWKRMEK KRSHLKRDTA
DQEDQVAPRL IAGKMTRRGD SPWQVVLLDS KKKLACGAVL
IHPSWVLTAA HCMDESKKLL VRLGEYDLRR WEKWELDLDI
KEVFVHPNYS KSTTDNDIAL LHLAQPATLS QTIVPICLPD
SGLAERELNQ AGQETLVTGW GYHSSREKEA KRNRTFVLNF
IKIPVVPHNE CSEVMSNMVS ENMLCAGILG DRQDACEGDS
GGPMVASFHG TWFLVGLVSW GEGCGLLHNY GVYTKVSRYL
DWIHGHIRDK EAPQKSWAP

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

A recitation of a range of values herein is merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments can become apparent to those of ordinary skill in the art upon reading the foregoing description. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Because many possible embodiments can be made of the invention without departing from the scope thereof, it is to be understood that all matter herein set forth is to be interpreted as illustrative, and not in a limiting sense.

1.-39. (canceled)

40. An auto-lytic recombinant thrombin comprising:
an amino acid sequence having at least 95 percent amino acid sequence identity to SEQ ID NO: 4; and
an amino acid substitution of glycine at position 48 of SEQ ID NO: 4.

41. The auto-lytic recombinant thrombin of claim 40, wherein the amino acid substitution of glycine at position 48 of SEQ ID NO: 4 is a non-conservative amino acid.

42. The auto-lytic recombinant thrombin of claim 41, wherein the amino acid substitution of glycine at position 48 of SEQ ID NO: 4 is a proline amino acid at position 48 of SEQ ID NO: 4.

43. The auto-lytic recombinant thrombin of claim 40, further comprising an amino acid substitution selected from the group consisting of a glutamic acid to alanine substitution at residue 40 of SEQ ID NO:4; an aspartic acid to alanine substitution at residue 47 of SEQ ID NO:4; a glutamic acid to alanine substitution at residue 52 of SEQ ID NO:4; a tryptophan to alanine substitution at residue 276 of SEQ ID NO:4; a glutamic acid to alanine substitution at residue 278 of SEQ ID NO:4; and combinations thereof.

44. The auto-lytic recombinant thrombin of claim 40, further comprising a tryptophan to alanine substitution at residue 276 of SEQ ID NO:4; a glutamic acid to alanine substitution at residue 278 of SEQ ID NO:4.

45. The auto-lytic recombinant thrombin of claim 40, further comprising a glutamic acid to alanine substitution at residue 40 of SEQ ID NO:4; an aspartic acid to alanine substitution at residue 47 of SEQ ID NO:4; a glutamic acid to alanine substitution at residue 52 of SEQ ID NO:4.

46. The auto-lytic recombinant thrombin of claim 40, further comprising a glutamic acid to alanine substitution at residue 40 of SEQ ID NO:4; an aspartic acid to alanine substitution at residue 47 of SEQ ID NO:4; a glutamic acid to alanine substitution at residue 52 of SEQ ID NO:4; a tryptophan to alanine substitution at residue 276 of SEQ ID NO:4; a glutamic acid to alanine substitution at residue 278 of SEQ ID NO:4.

47. The auto-lytic recombinant thrombin of claim 40, wherein the auto-lytic recombinant thrombin precursor is unglycosylated.

48. The auto-lytic recombinant thrombin of claim 40, wherein the auto-lytic recombinant thrombin precursor is glycosylated.

49. The auto-lytic recombinant thrombin of claim 40, further comprising a tag.

50. The auto-lytic recombinant thrombin of claim 49, wherein the tag is selected from the group consisting of a FLAG peptide, β-galactosidase, glutathione-S-transferase, a histidine tag, a chitin binding protein, a maltose binding protein, a V5 tag, a c-myc tag, a HA-tag, and combinations thereof.
